SEQUENCE LISTING (5 sequences)

SEQ ID NO: 1 - RNA, 21 nt, synthetic construct
  source 1..21: mol_type = other RNA
gctgggaaggcaaagggacgt

SEQ ID NO: 2 - RNA, 18 nt, synthetic construct
  source 1..18: mol_type = other RNA
ttcccagccaacgcacca

SEQ ID NO: 3 - RNA, 21 nt, synthetic construct
  source 1..21: mol_type = other RNA
cgggggcggggccgaagcgcg

SEQ ID NO: 4 - RNA, 20 nt, synthetic construct
  source 1..20: mol_type = other RNA
gggctggggcgcggggaggt

SEQ ID NO: 5 - RNA, 24 nt, synthetic construct
  source 1..24: mol_type = other RNA
ggaggccggggtggggcggggcgg

(19) United States
(12) Patent Application Publication
(10) Pub. No.: US 2025/0144129 A1
(43) Pub. Date: May 8, 2025

(54) PHARMACEUTICAL COMPOSITIONS FOR PREVENTING OR TREATING LIVER DISEASES WITH FIBROSIS

(71) Applicants: SHIBUYA CORPORATION, Ishikawa (JP); YAMAGUCHI UNIVERSITY, Yamaguchi (JP)

(72) Inventors: Naoki YAMAMOTO, Yamaguchi (JP); Taro TAKAMI, Yamaguchi (JP); Toshihiko MATSUMOTO, Yamaguchi (JP); Daiki KAWAMOTO, Yamaguchi (JP)

(73) Assignees: SHIBUYA CORPORATION, Ishikawa (JP); YAMAGUCHI UNIVERSITY, Yamaguchi (JP)

(21) Appl. No.: 18/939,823

(22) Filed: Nov. 7, 2024

(30) Foreign Application Priority Data
Nov. 7, 2023 (JP) 2023-189942
Sep. 24, 2024 (JP) 2024-164983

(51) Int. Cl. A61K 31/7088; A61K 47/69; A61P 1/16

(52) U.S. Cl. CPC A61K 31/7088; A61K 47/6909; A61P 1/16

(57) ABSTRACT

It is an object of the present invention to provide a drug for preventing or treating a liver disease associated with fibrosis, the drug containing microRNAs as an active ingredient. A pharmaceutical composition for preventing or treating a liver disease associated with fibrosis, the pharmaceutical composition containing at least one kind of microRNAs selected from miR 1237-5p, miR 204-3p, miR 7977, miR 6089, and miR 5787 as an active ingredient, is produced.

### TECHNICAL FIELD

The present invention relates to a pharmaceutical composition for preventing or treating a liver disease associated with fibrosis, an extracellular vesicle (Extracellular Vesicle: EV), a culture product, and a method for producing extracellular vesicles for suppressing liver fibrosis.

### BACKGROUND ART

Fibrosis of the liver (liver fibrosis) mainly occurs in conjunction with inflammation caused by hepatitis viruses or alcohol, and is likely to occur particularly when inflammation has become chronic. It is known that chronic hepatitis or liver cirrhosis associated with liver fibrosis is likely to develop hepatocellular carcinoma. For viral liver cirrhosis or viral liver cancer, promising antiviral drugs have been developed so far; however, these drugs do not completely cure those diseases.

In recent years, along with changes in lifestyle, there has been an increase in liver disorders, precancerous lesions such as hepatitis or liver cirrhosis, and liver cancer, which are caused by chronic metabolic disorders due to excessive alcohol consumption, overeating, and lack of exercise. Liver cirrhosis or liver cancer caused by metabolic disorders has become a social problem because these diseases are related to lifestyle-related diseases such as obesity and diabetes. However, it is the current situation that symptomatic treatment such as diet and exercise therapy is still the only treatment method available.

The inventors of the present invention have hitherto conducted research on suppressants for the fibrosis of liver and have disclosed a suppressant for the fibrosis of the liver, containing dantrolene or a pharmacologically acceptable salt thereof as an active ingredient (see Patent Document 1).

In recent years, a method for diagnosing cancer by measuring microRNA has been developed. MicroRNA (hereinafter, also referred to as “miRNA or miR”) is RNA that is present in cells and has a length of about 18 to 25 bases, and is a type of non-coding RNA that is believed to have a function of regulating the expression of other genes. To date, markers for colorectal cancer diagnosis formed of microRNA molecules (see Patent Document 2) have been disclosed. Furthermore, exosomes containing predetermined microRNAs have been disclosed for the purpose of treating various diseases (see Patent Document 3). In addition, in regard to microRNA and liver fibrosis, it has been disclosed that miR 6848-5p and miR 6848-3p are involved in liver fibrosis (see Patent Document 4). However, sufficient research has not yet been conducted on the relationship between microRNA and fibrosis in the liver, and it is the current situation that microRNA has not yet been put to practical use.

### CITATION LIST

### Patent Document

Patent Document 1: JP 2020-007237 A
Patent Document 2: WO 2021/033605
Patent Document 3: JP 2022-538004 A
Patent Document 4: WO 2022/270071

### SUMMARY OF THE INVENTION

It is an object of the present invention to provide a pharmaceutical composition for preventing or treating a liver disease associated with fibrosis, the pharmaceutical composition including microRNA or a precursor thereof as an active ingredient.

The present inventors have conducted research on liver regenerative therapy, particularly liver cirrhosis regenerative therapy, using mesenchymal stem cells. While searching for a method for producing mesenchymal stem cells optimized for the regeneration of a liver that has become cirrhotic, the present inventors found that predetermined microRNAs are deeply involved in suppressing liver fibrosis, thus completing the present invention.

That is, the present invention is as follows.

[1] A pharmaceutical composition for preventing or treating a liver disease associated with fibrosis, the pharmaceutical composition including at least one kind of microRNAs selected from miR 1237-5p, miR 204-3p, miR 7977 miR 6089, and miR 5787, or precursors thereof as an active ingredient.

[2] The pharmaceutical composition according to the above-described item [1], further including a vehicle for holding the microRNAs or precursors thereof.

[3] The pharmaceutical composition according to the above-described item [2], wherein the vehicle for holding the microRNAs or precursors thereof is any one extracellular vesicle selected from an exosome, a microvesicle, a liposome (lipid bilayer), and an apoptotic vesicle.

[4] The pharmaceutical composition according to the above-described item [3], wherein the extracellular vesicle is a mesenchymal stem cell-derived extracellular vesicle.

[5] The pharmaceutical composition according to the above-described item [3] or [4], wherein the extracellular vesicle is a cell-derived extracellular vesicle obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed in the cells.

[6] The pharmaceutical composition according to the above-described item [1] or [2], which is used in combination with mesenchymal stem cells to be administered to a target.

[7] An extracellular vesicle derived from cells and obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed in the cells, the extracellular vesicle including at least one kind of microRNAs selected from miR 1237-5p, miR 204-3p, miR 7977, miR 6089, and miR 5787, or precursors thereof.

[8] The extracellular vesicle according to the above-described item [7], wherein the extracellular vesicle is a mesenchymal stem cell-derived extracellular vesicle.

[9] A culture product obtained by introducing a polynucleotide encoding at least one kind of microRNAs selected from extraneous miR 1237-5p, miR 204-3p, miR 7977, miR 6089, and miR 5787, or precursors thereof into cells, allowing the at least one kind of microRNAs or precursors thereof to be expressed in the cells, and culturing the cells.

[10] A method for producing extracellular vesicles for suppressing liver fibrosis, which contains at least one kind of microRNAs selected from extraneous miR 1237-5p, miR 204-3p, miR 7977, miR 6089, and miR 5787, or precursors thereof,
the method including the following steps A to C:
step A of introducing a polynucleotide encoding at least one kind of microRNAs selected from extraneous miR 1237-5p, miR 204-3p, miR 7977, miR 6089, and miR 5787, or precursors thereof into mesenchymal stem cells;
step B of culturing the mesenchymal stem cells obtained in step A to obtain a culture product; and
step C of harvesting extracellular vesicles from the culture product.

Furthermore, other embodiments of the present invention include: (I) a method for preventing or treating a liver disease associated with fibrosis, the method including administering an effective amount of at least one kind of microRNAs selected from miR 1237-5p, miR 204-3p, miR 7977, miR 6089, and miR 5787, or precursors thereof to a target in need of the microRNA or the precursor; (II) use of at least one kind of microRNAs selected from miR 1237-5p, miR 204-3p, miR 7977, miR 6089, and miR 5787, or precursors thereof, and a polynucleotide encoding the microRNA or the precursor, for the production of a pharmaceutical composition for preventing or treating liver disease associated with fibrosis; and (III) at least one kind of microRNAs selected from miR 1237-5p, miR 204-3p, miR 7977, miR 6089, and miR 5787, or precursors thereof, and a polynucleotide encoding the microRNA or the precursor, for use in the prevention or treatment of a liver disease associated with fibrosis. Here, the term “effective amount” means an amount required for administration to expect a prophylactic or therapeutic effect in a target. Therefore, the effective amount can be adjusted depending on the degree of severity of the disease of the target of administration, the dosage form to be administered, the age, body weight, and gender of the target of administration, the administration time, the route of administration, the duration of treatment, and the like.

When the pharmaceutical composition of the present disclosure is used, it is possible to prevent or treat a liver disease associated with fibrosis.

### BRIEF DESCRIPTION OF DRAWINGS

FIG. 1 is a diagram showing the results of investigating the expression of ACTA2, COL1A1, and ELN using a group of 11 types of miRNAs and groups of 10 types of miRNAs in Example 4, in order to select candidates for miRNA that are effective in suppressing fibrosis.

FIG. 2 is a diagram showing the results of investigating the expression of ACTA2, COL1A1, and ELN using a group of 5 types of miRNAs and groups of 4 types of miRNAs in Example 5, in order to select candidates for miRNA that are effective in suppressing fibrosis.

FIG. 3A is a diagram showing the results of evaluating ACTA2, which is a fibrosis marker, by a real-time-PCR method in Example 6. The vertical axis shows the relative ratios of a TGF-β group, a 5-kind miRNAs group, and single miRNA groups when the Fold change of the control group is taken as 1.0 (the same applies to the following FIG. 3B to FIG. 3E).

FIG. 3B is a diagram showing the results of evaluating COL1A1, which is a fibrosis marker, by the real-time-PCR method in Example 6.

FIG. 3C is a diagram showing the results of evaluating COL1A2, which is a fibrosis marker, by the real-time-PCR method in Example 6.

FIG. 3D is a diagram showing the results of evaluating COL3A1, which is a fibrosis marker, by the real-time-PCR method in Example 6.

FIG. 3E is a diagram showing the results of evaluating ELN, which is a fibrosis marker, by the real-time-PCR method in Example 6.

FIG. 4 is images showing the results of evaluating the area of fibrosis in cirrhotic mice using the 5-kind miRNAs group and the control group in Example 7.

FIG. 5 is a diagram showing the results of evaluating fibrosis markers ACTA2, COL1A1, and ELN by the real-time-PCR method when mesenchymal stem cell (MSC)-derived extracellular vesicles (EVs) were administered, in Example 8.

### DESCRIPTION OF EMBODIMENTS

The pharmaceutical composition for preventing or treating a liver disease associated with fibrosis in the present specification is not particularly limited as long as it is a pharmaceutical composition for preventing or treating a liver disease associated with fibrosis, which includes at least one kind of microRNAs selected from miR 1237-5p, miR 204-3p, miR 7977, miR 6089, and miR 5787 (hereinafter, also referred to as “present microRNA”), or precursors thereof as an active ingredient, and in the following description, the pharmaceutical composition will also be referred to as “present pharmaceutical composition”. Furthermore, the extracellular vesicle in the present specification is not particularly limited as long as it is a cell-derived extracellular vesicle obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed in the cells, the extracellular vesicle including at least one kind of microRNAs selected from miR 1237-5p, miR 204-3p, miR 7977, miR 6089, and miR 5787, or precursors thereof, and in the following description, the extracellular vesicle will also be referred to as “present extracellular vesicle”. In addition, the culture product in the present specification is not particularly limited as long as it is a culture product obtained by introducing a polynucleotide encoding at least one kind of microRNAs selected from extraneous miR 1237-5p, miR 204-3p, miR 7977, miR 6089, and miR 5787, or precursors thereof into cells, allowing the microRNAs or precursors thereof to be expressed in the cells, and culturing the cells, and in the following description, the culture product will also be referred to as “present culture product”. Furthermore, the method for producing extracellular vesicles (Extracellular Vesicles: EVs) for suppressing liver fibrosis in the present specification is not particularly limited as long as it is a method for producing extracellular vesicles for suppressing liver fibrosis, which contains at least one kind of microRNAs selected from extraneous miR 1237-5p, miR 204-3p, miR 7977, miR 6089, and miR 5787, or precursors thereof, the method including the following steps A to C: step A of introducing a polynucleotide encoding at least one kind of microRNAs selected from extraneous miR 1237-5p, miR 204-3p, miR 7977, miR 6089, and miR 5787, or precursors thereof into mesenchymal stem cells; step B of culturing the mesenchymal stem cells obtained in step A to obtain a culture product; and step C of harvesting extracellular vesicles from the culture product, and in the following description, the method will also be referred to as “present method for producing extracellular vesicles”.

Here, the above-described present microRNA may be a single-stranded mature microRNA, or may be a double-stranded microRNA having the above-described single-stranded mature microRNA and a base sequence complementary to the mature microRNA. Among the present microRNAs, the polynucleotides of the single-stranded mature microRNAs each have the sequences shown in the following Table 1. Furthermore, the numbers in the parentheses are the ID and Accession No. in the miR Base (https://mirbase.org/).

TABLE 1

| | | | | miRBase |
|---|---|---|---|---|
| | miRBase | Base | | Accession |
| miRNA | ID | sequence | | No. |
| miR-204- | hsa-mir- | 5′-GCUG | (SEQ ID | MIMAT0022693 |
| 3p | 204-3p | GGAAGGC | NO: 1) | |
| | | AAAGGGA | | |
| | | CGU-3′ | | |
| miR-7977 | hsa-miR- | 5′-UUCC | (SEQ ID | MIMAT0031180 |
| | 7977 | CAGCCAA | NO: 2) | |
| | | CGCACCA | | |
| | | -3′ | | |
| miR-1237- | hsa-miR- | 5′-CGGG | (SEQ ID | MIMAT0022946 |
| 5p | 1237-5p | GGCGGGG | NO: 3) | |
| | | CCGAAGC | | |
| | | GCG-3′ | | |
| miR-5787 | hsa-miR- | 5′-GGGC | (SEQ ID | MIMAT0023252 |
| | 5787 | UGGGGCG | NO: 4) | |
| | | CGGGGAG | | |
| | | GU-3′ | | |
| miR-6089 | hsa-miR- | 5′-GGAG | (SEQ ID | MIMAT0023714 |
| | 6089 | GCCGGGG | NO: 5) | |
| | | UGGGGGG | | |
| | | GGGGGG- | | |
| | | 3′ | | |

Furthermore, the present microRNA also includes a mutant of a single-stranded mature microRNA shown in the following (a) or (b).
(a) A polynucleotide in which one or several bases have been deleted, added, or substituted in a base sequence set forth in any one of SEQ ID NO:1 to SEQ ID NO: 5, the polynucleotide resulting in a transcriptional product having a liver fibrosis suppressive action.
(b) A polynucleotide having a sequence identity of 80% or higher, preferably 85% or higher, more preferably 90% or higher, even more preferably 95% or higher, and most preferably 98% or higher, with a base sequence set forth in any one of SEQ ID NO:1 to SEQ ID NO: 5, the polynucleotide resulting in a transcriptional product having a liver fibrosis suppressive action.

With regard to the above-described (a) or (b), whether the polynucleotide has a liver fibrosis suppressive action can be checked by, for example, using a polynucleotide in which one or several bases have been deleted, added, or substituted in a base sequence set forth in any one of SEQ ID NO: 1 to SEQ ID NO: 5, or a polynucleotide having a sequence identity of 80% or higher, preferably 85% or higher, more preferably 90% or higher, even more preferably 95% or higher, and most preferably 98% or higher, with a base sequence set forth in any one of SEQ ID NO:1 to SEQ ID NO: 5, introducing the polynucleotide to be evaluated into hepatic stellate cells (HHSteC) or the like according to the method described in Examples that will be described below, culturing the cells, and examining whether the expression of a fibrosis marker is suppressed. As the above-described fibrosis marker, one kind, preferably two kinds, more preferably three kinds, and particularly preferably four kinds, selected from ACTA2, COL1A1, ELN, COL1A2, and COL3A1 may be evaluated. A combination of two kinds may be the combination of COL1A2 and COL3A1, a combination of three kinds may be the combination of ACTA2, COL1A2, and COL3A1, or the combination of COL1A2, COL3A1, and ELN, and a combination of four kinds may be the combination of ACTA2, COL1A1, COL1A2, and COL3A1.

The above-described expression “one or several” is preferably 1 to 5, more preferably 1 to 4, even more preferably 1 to 3, particularly preferably 1 or 2, and most preferably 1. Furthermore, it is preferable that a seed sequence consisting of 6 or 7 bases from the 2nd to 7th or 8th bases as counted from the 5′-end in the base sequence set forth in any one of SEQ ID NO:1 to SEQ ID NO: 5 is retained. That is, regarding (a), (a)′ a polynucleotide in which the seed sequence in the base sequence set forth in any one of SEQ ID NO:1 to SEQ ID NO: 5 is retained, one or several bases have been deleted, added, or substituted in the base sequence set forth in any one of SEQ ID NO:1 to SEQ ID NO: 5, the polynucleotide resulting in a transcriptional product having a liver fibrosis suppressive action, may be mentioned, and regarding (b), (b)′ a polynucleotide in which the seed sequence in the base sequence set forth in any one of SEQ ID NO: 1 to SEQ ID NO: 5 is retained, and which has a sequence identity of 80% or higher with the base sequence set forth in any one of SEQ ID NO: 1 to SEQ ID NO: 5, the polynucleotide resulting in a transcriptional product having a liver fibrosis suppressive action, may be mentioned.

The above-described precursor of a microRNA means an RNA that has a stem-loop structure, contains the above-described single-stranded mature microRNA or a mutant thereof in the precursor strand, and can produce a mature miRNA by cutting or double-strand cleavage. Specific examples thereof include pri-miRNA, pre-miRNA, and a double-stranded miRNA consisting of a mature miRNA and an antisense strand thereof. The length of the precursor of the microRNA may be 50 to 100 nt, and preferably 65 to 85 nt.

In the above-described microRNAs or precursors thereof, in order to improve stability, any number of bases, for example, 1 to 15 bases, 1 to 10 bases, 1 to 5 bases, or 1 to 3 bases may be added to the 5′-end and/or the 3′-end. In order to increase the affinity with the complementary strand nucleic acid, the microRNA or the precursor may be a modified analogue such as a sugar moiety-modified nucleotide analogue or a phosphodiester bond-modified nucleotide analogue, or a nucleic acid derivative. In addition, the above-described present microRNA may be a microRNA mimic, which is a double-stranded RNA with a chemically modified passenger strand.

The above-described sugar moiety-modified nucleotide analogue may be any nucleotide analogue in which any chemical structural substance has been added to or substituted with a portion or the entirety of the chemical structure of the nucleotide sugar. Specific examples of the sugar moiety-modified nucleotide analogue include a nucleotide analogue substituted with 2′-O-methylribose, a nucleotide analogue substituted with 2′-O-propylribose, a nucleotide analogue substituted with 2′-methoxyethoxyribose, a nucleotide analogue substituted with 2′-O-methoxyethylribose, a nucleotide analogue substituted with 2′-O-[2-(guanidium)ethyl]ribose, a nucleotide analogue substituted with 2′-fluororibose, a bridged structure type artificial nucleic acid (Bridged Nucleic Acid) (BNA) having two cyclic structures by introducing a bridged structure into a sugar moiety, more specifically, a locked artificial nucleic acid (Locked Nucleic Acid) (LNA) in which an oxygen atom at the 2′-position and a carbon atom at the 4′-position are crosslinked through methylene, and an ethylene bridged structure type artificial nucleic acid (Ethylene bridged nucleic acid) (ENA) [Nucleic acid Research, 32, e175 (2004)], and further examples include a peptide nucleic acid (PNA) [Acc. Chem. Res., 32, 624 (1999)], an oxypeptide nucleic acid (OPNA) [J. Am. Chem. Soc., 123, 4653 (2001)], and a peptide ribonucleic acid (PRNA) [J. Am. Chem. Soc., 122, 6900 (2000)].

The above-described phosphodiester bond-modified nucleotide analogue may be any nucleotide analogue in which any chemical substance has been added to or substituted with a portion or the entirety of the chemical structure of a phosphodiester bond of a nucleotide. Specific examples of the phosphodiester bond-modified nucleotide analogue include a nucleotide analogue substituted with a phosphorothioate bond, and a nucleotide analogue substituted with an N3′-P5′ phosphoramidate bond [Cell Engineering, 16, 1463-1473 (1997)] [RNAi Method and Antisense Method, Kodansha (2005)].

The above-described nucleic acid derivative may be any molecule as long as it is a molecule obtained by adding another chemical substance to a nucleic acid in order to improve the nuclease resistance of the nucleic acid as compared to the nucleic acid, in order to stabilize the nucleic acid, in order to increase the affinity to the complementary strand nucleic acid, in order to increase the cell permeability, or in order to visualize the nucleic acid. Specific examples thereof include a 5′-polyamine adduct, a cholesterol adduct, a steroid adduct, a bile acid adduct, a vitamin adduct, a Cy5 adduct, a Cy3 adduct, a 6-FAM adduct, and a biotin adduct.

The polynucleotide encoding the present microRNAs or precursors thereof may be incorporated into an expression vector. The expression vector may be an expression vector that can be introduced into cells when brought into contact with the cells, and can express the predetermined present microRNAs or precursors thereof encoded by polynucleotides encoding the present microRNAs or precursors thereof in the cells, and specifically, the viral vectors or non-viral vectors that will be described below may be mentioned.

The origin of the above-described present microRNA is not particularly limited, and examples thereof include human, monkey, pig, dog, cat, horse, sheep, mouse, and rabbit.

In the present specification, the term “fibrosis” in the liver refers to excessive formation of fibrous connective tissue occurring due to various causes such as non-alcoholic steatohepatitis, persistent infection with hepatitis viruses, excessive alcohol intake, auto-immunological mechanism, intrahepatic cholestasis, drug-induced causes, abnormal metal metabolism, and congestive liver, and occurring as the balance between destruction and regeneration of liver cells is disrupted, fibrosis is induced in necrotic tissue, and extracellular matrix such as collagen deposits in the liver parenchyma. The hepatitis that causes fibrosis is not particularly limited, and examples thereof include non-alcoholic steatohepatitis; viral hepatitis such as hepatitis B and hepatitis C; chronic hepatitis; acute hepatitis; liver cirrhosis; and hepatitis associated with liver cancer.

The liver disease according to the present specification is not particularly limited as long as it is a liver disease associated with fibrosis, and examples thereof include chronic hepatitis B, chronic hepatitis C, non-alcoholic fatty liver diseases (NAFLD; also referred to as MASLD (metabolic dysfunction-associated steatotic liver disease)) such as non-alcoholic steatohepatitis (NASH; also referred to as MASH (metabolic dysfunction-associated steatohepatitis)) and non-alcoholic fatty liver (NAFL), metabolic dysfunction-associated steatotic liver diseases (MASLD), autoimmune diseases, liver cirrhosis, and hepatocellular carcinoma.

In the present specification, the term “prevention” means preventing or delaying the onset of a disease by administering the present pharmaceutical composition to a target before the onset of the disease, but does not necessarily mean complete suppression of the onset. Specifically, the term “prevention” means that the progress of fibrosis in the liver is suppressed or delayed by administering the present pharmaceutical composition to a target. Incidentally, the onset of a disease means that symptoms of the disease appear in the body.

In the present specification, the term “treatment” means that symptoms of a disease are mitigated by administering the present pharmaceutical composition to a target after the onset of the disease, but does not necessarily mean complete suppression of the symptoms of the disease. Specifically, when the pathological state of fibrosis in the liver in the disease, or one or more of the biological symptoms of that pathological state can be improved by administering the present pharmaceutical composition, it can be said that a liver disease associated with fibrosis can be treated using this pharmaceutical composition.

The present pharmaceutical composition may include a vehicle for holding microRNAs or precursors thereof. The vehicle for holding microRNAs or precursors thereof may be any vehicle capable of holding microRNAs or precursors thereof. Here, holding microRNAs or precursors thereof may refer to a state in which all or some of microRNAs or precursors thereof are enclosed, adhered, or embedded.

Examples of this vehicle for holding microRNAs or precursors thereof include an extracellular vesicle such as an exosome, a microvesicle, a liposome (lipid bilayer), or an apoptotic vesicle; a micelle; ionized lipids; and non-cationic lipids, and the vehicle is preferably an exosome.

The above-described exosome is released from most cell types, can be found in many body fluids, and encloses a nucleic acid such as microRNA or mRNA, or a protein. In addition, in another preferred embodiment, the exosome may be an exosome that is positive for at least one of cellular exosome markers selected from the group consisting of CD (cluster of differentiation) 9, CD81, CD63, CD41a, CD41b, CD42b, CD61, CD62P, and syntenin.

Regarding the size of the exosome, the average particle size may be a diameter of 10 to 1000 nm, preferably 20 to 200 nm, and more preferably 30 to 140 nm. The average particle size can be measured by a dynamic light scattering method, observation with an optical interference microscope, or observation with an electron microscope.

The exosome according to the present invention is preferably a mesenchymal stem cell-derived exosome. The mesenchymal stem cell-derived exosome is preferable from the viewpoint of having an anti-inflammatory action and an effect for promoting wound healing, from the viewpoint that the exosome is easily harvested because mesenchymal stem cells can be cultured without serum, and from the viewpoint that since mesenchymal stem cells have almost no risk of becoming cancerous, it is suggested that exosomes thereof are also highly safe, and that those exosomes enclose various growth factors.

Furthermore, the origin of the cells used for preparing the above-described exosomes is not limited to human and may also be other mammals (for example, mouse, rat, rabbit, dog, cat, monkey, sheep, cow, and horse).

The term “mesenchymal stem cells” according to the present specification means a population of stem cells, or precursor cells thereof, capable of differentiation into all or some of the cells of the mesenchymal lineage, such as osteoblasts, chondroblasts, and lipoblasts. More specifically, dental pulp stem cells, bone marrow-derived stem cells, adipose tissue-derived stem cells, and the like may be mentioned. Furthermore, the above-described mesenchymal stem cells may also be derived from induced pluripotent stem cells (iPS cells). When iPS cell-derived mesenchymal stem cells are used, it is possible to maintain stable quality without relying on the donor.

The present extracellular vesicle is an extracellular vesicle derived from cells, obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed in the cells, and here, the cells in which the microRNAs or precursors thereof are expressed express the above-described at least one kind of microRNAs or precursors thereof excessively as compared with natural cells, for example, express the microRNAs or precursors thereof twofold, threefold, or fivefold or more. The method of introducing a polynucleotide encoding at least one kind of microRNAs selected from extraneous miR 1237-5p, miR 204-3p, miR 7977, miR 6089, and miR 5787 into cells is not particularly limited, and a method of introducing a polynucleotide encoding a microRNA by a known genetic engineering technique may be mentioned. Specifically, transfection using a viral vector, electroporation using a non-viral vector, lipofection, and microinjection may be mentioned.

Examples of the above-described viral vector include viral vectors such as an adenovirus vector, a retrovirus vector, an adeno-associated virus vector, a poxvirus, a vaccinia virus vector, a baculovirus vector, and a herpes virus vector. Furthermore, examples of the non-viral vector include a plasmid vector, a phagemid vector, a cosmid vector, and a bacteriophage.

The extracellular vesicle according to the present specification may be a cell-derived extracellular vesicle obtained by introducing polynucleotides encoding two kinds of extraneous microRNAs or precursors thereof into cells, and allowing the microRNAs or precursors thereof to be expressed therein. Combinations of the two kinds of extraneous microRNAs include miR 1237-5p and miR 204-3p; miR 1237-5p and miR 7977; miR 1237-5p and miR 6089; miR 1237-5p and miR 5787; miR 204-3p and miR 7977; miR 204-3p and miR 6089; miR 204-3p and miR 5787; miR 7977 and miR 6089; miR 7977 and miR 5787; and miR 6089 and miR 5787.

The extracellular vesicle according to the present specification may be a cell-derived extracellular vesicle obtained by introducing polynucleotides encoding three kinds of extraneous microRNAs or precursors thereof into cells, and allowing the microRNAs or precursors thereof to be expressed therein. Combinations of the three kinds of extraneous microRNAs include miR 1237-5p, miR 204-3p, and miR 7977; miR 1237-5p, miR 204-3p, and 6089; miR 1237-5p, miR 204-3p, and miR 5787; miR 1237-5p, miR 7977, and miR 6089; miR 1237-5p, miR 7977, and miR 5787; miR 1237-5p, miR 6089, and miR 5787; miR 204-3p, miR 7977, and miR 6089; miR 204-3p, miR 7977, and miR 5787; miR 204-3p, miR 6089, and miR 5787; and miR 7977, miR 6089, and miR 5787.

The extracellular vesicle according to the present specification may be a cell-derived extracellular vesicle obtained by introducing polynucleotides encoding four kinds of extraneous microRNAs or precursors thereof into cells, and allowing the microRNAs or precursors thereof to be expressed therein. Combinations of the four kinds of extraneous microRNAs include miR 1237-5p, miR 204-3p, miR 7977, and miR 6089; miR 1237-5p, miR 204-3p, miR 7977, and miR 5787; miR 1237-5p, miR 204-3p, miR 5787, and miR 6089; miR 1237-5p, miR 7977, miR 6089, and miR 5787; and miR 204-3p, miR 7977, miR 6089, and miR 5787.

The extracellular vesicle according to the present specification may be a cell-derived extracellular vesicle obtained by introducing polynucleotides encoding five kinds of extraneous microRNAs or precursors thereof into cells, and allowing the microRNAs or precursors thereof to be expressed therein. A combination of the five kinds of extraneous micrRNAs may be miR 1237-5p, miR 204-3p, miR 7977, miR 6089, and miR 5787.

The present pharmaceutical composition may be used in combination with mesenchymal stem cells, or may be used in combination with another medicine for preventing or treating a liver disease associated with fibrosis. Regarding the above-described method of “using in combination with mesenchymal stem cells”, a method of treating using mesenchymal stem cells and then using the present pharmaceutical composition, a method of simultaneously using the present pharmaceutical composition and mesenchymal stem cells, or a method of treating using the present pharmaceutical composition and then using mesenchymal stem cells, may be mentioned. Similarly, regarding the above-describe method of “using in combination with another medicine for preventing or treating a liver disease associated with fibrosis”, a method of treating using another medicine for preventing or treating a liver disease associated with fibrosis, and then using the present pharmaceutical composition, a method of simultaneously using the present pharmaceutical composition and another medicine for preventing or treating a liver disease associated with fibrosis, or a method of treating using the present pharmaceutical composition, and then using another medicine for preventing or treating a liver disease associated with fibrosis, may be mentioned.

The term “culture product” in the present culture product means a product obtained by introducing a polynucleotide encoding at least one kind of microRNAs selected from extraneous miR 1237-5p, miR 204-3p, miR 7977, miR 6089, and miR 5787, or precursors thereof into cells, allowing the at least one kind of microRNAs or precursors thereof to be expressed in the cells, and culturing the cells in a known liquid medium or solid medium, the culture product being in a state of including the cultured cells. A culture fluid can be obtained by removing the above-described cells from the above-described culture product. This culture fluid contains extracellular vesicles derived from the cultured cells. Then, the extracellular vesicles will include at least one kind of microRNAs selected from miR 1237-5p, miR 204-3p, miR 7977, miR 6089, and miR 5787.

The medium used during the above-described culturing can be appropriately regulated in accordance with the cells to be cultured, and for example, in the case of mesenchymal stem cells, Dulbecco's Modified Eagle Medium (DMEM), Minimum Essential Medium (MEM), Basal Medium Eagle (BME), and StemFit (registered trademark) For Mesenchymal Stem Cell (MSC) may be mentioned.

Extracellular vesicles can be harvested from the present culture product. Regarding such a method, an ultracentrifugation method of applying the medium to a centrifuge, and removing cells and foreign matter to harvest the supernatant, a polymer sedimentation method, an immunoprecipitation method, and a method of using a commercially available kit for harvesting extracellular vesicles may be mentioned. In this ultracentrifugation method, extracellular vesicles can be harvested by performing centrifugation in advance under the conditions of 2 to 60 minutes at 500 to 3500 rpm and then performing ultracentrifugation (for example, 100000 to 1000000 G, 30 minutes to 12 hours). Thereafter, the extracellular vesicles may be purified by filtration using a filter or the like.

The present pharmaceutical composition may include suitable diluents, buffer agents, suspending agents, dispersants, preservatives, stabilizers, and excipients that are commonly used for the manufacture of drugs or pharmaceutical compositions. Furthermore, the present pharmaceutical composition can be prepared in the form of a parenteral preparation such as an injectable preparation or a drip infusion preparation, or an oral preparation.

The present pharmaceutical composition can be administered via a variety of routes including subcutaneous, intramuscular, intravenous, transdermal, or peroral routes. A preferable dosage can be appropriately determined by those skilled in the art, in the light of various relevant factors such as the age, gender, and body weight of the patient, the health condition, and the degree of severity of the disease. Specifically, the dosage in terms of the weight of the microRNAs or precursors thereof as the active ingredient may be 0.01 to 500 mg/60 kg of body weight, and preferably 0.1 to 10 mg/60 kg of body weight, per day. Furthermore, the dosage of the extracellular vesicles may be 10 mg to 25 g/60 kg of body weight, and preferably 100 mg to 5 g/60 kg of body weight, per day.

The target of administration of the present pharmaceutical composition may be a patient who has been diagnosed to have a liver disease associated with fibrosis, or a patient who has been diagnosed to be suspected of having a liver disease associated with fibrosis.

The contents described in all the patent documents and non-patent documents cited in the present specification are incorporated herein by reference in their entirety.

Hereinafter, the present invention will be more specifically described by way of Examples; however, the technical scope of the present invention is not intended to be limited to these examples.

### [Example 1] miRNA Highly Expressed in Mesenchymal Stem Cell (MSC)-Derived Extracellular Vesicles (EVs)

First, among the 2632 types of miRNA, miRNAs that were highly expressed in each extracellular vesicle were identified by array analysis, using commercially available MSC-derived extracellular vesicles and MSC-derived extracellular vesicles from liver cirrhosis cases.

The MSC-derived extracellular vesicles (MSC-EVs) from liver cirrhosis cases were harvested by the following method.
MSC cells from three patients with liver cirrhosis cases were cultured for 2 days in a partially modified medium based on StemFit For Mesenchymal Stem Cell (Ajinomoto Co., Inc.).
Medium exchange (DMEM+F12 medium: Thermo Fisher Scientific Inc.) was performed, the MSC cells were cultured for 2 days, and then the culture supernatant was harvested.
The harvested culture supernatant was centrifuged at 500 G for 5 minutes at 4° C., and then the supernatant was harvested.
The supernatant was further centrifuged at 2000 G for 30 minutes at 4° C., and then the supernatant was harvested.
The harvested supernatant was filtered through a 0.22-μm filter made of PVDF.
To the filtered liquid, an equal amount of 16% PEG+NaCl was added, and the mixture was left to stand overnight at 4° C.
Next day, the liquid that had been left to stand overnight was centrifuged at 3300 G for 60 minutes at 4° C., and then the supernatant was removed.
1 mL of phosphate buffered saline (PBS) was added thereto, and the mixture was subjected to ultracentrifugation at 100000 G for 70 minutes at 4° C.
The supernatant was removed, an appropriate amount of PBS was added to the residue, the mixture was mixed by shaking for about 30 minutes, and MSC-EVs from liver cirrhosis cases were obtained as an extracellular vesicle suspension.

Next, array analysis was performed based on commercially available MSC-EVs of three healthy persons (a 26-year old male, a 29-year old female, and a 30-year old male) and the above-described MSC-EVs of three patients with liver cirrhosis cases. Microarray analysis was performed by treating the MSC-EVs with QIAzol Lysis Reagent (QIAGEN) and using an oligo DNA probe for microRNA detection (Toray Industries, Inc.). Whether the expression of miRNA was high was judged using the signal intensity (1000 or higher) after normalization by global normalization.

As a result, 39 kinds, 52 kinds, and 63 kinds of miRNAs were identified as miRNAs highly expressed in the commercially MSC-EVs of three healthy persons, respectively, and among them, 39 kinds of miRNAs were obtained as miRNAs highly expressed in all the three persons. Furthermore, 45 kinds, 79 kinds, and 79 kinds of miRNAs were identified as miRNAs highly expressed in the MSC-EVs of the above-described three patients with liver cirrhosis cases, and 40 kinds of miRNAs were obtained as miRNAs highly expressed in all the three patients. Further, 36 kinds of miRNAs were identified as miRNAs that were highly expressed in common in the 39 kinds obtained by array analysis of the commercially available MSC-EVs and the 40 kinds obtained by array analysis of the MSC-EVs of the liver cirrhosis cases.

### [Example 2] Selection of miRNAs Effective for Suppressing Fibrosis

36 kinds of miRNAs highly expressed in the MSC-derived EVs of healthy persons and liver cirrhosis cases were identified by Example 1. Next, among these, miRNAs that were intracellularly lowly expressed in hepatic stellate cells (HHSteC), which caused fibrosis when activated, were investigated.

The intracellular expression of miRNAs in hepatic stellate cells (HHSteC) was carried out using microarray. The microarray analysis was performed by treating the HHSteC with RNeasy (registered trademark) Mini Kit (QIAGEN) and using an oligo-DNA probe for microRNA detection (Toray Industries, Inc.). Whether a miRNA was highly expressed was judged from the signal intensity (1000 or higher) after normalization by global normalization.

As a result of microarray analysis, it was verified that among the 36 kinds of miRNAs highly expressed in the MSC-derived EVs of healthy persons and liver cirrhosis cases obtained in Example 1, 11 types of miRNAs (miR-1908-5p, miR-204-3p, miR-3960, miR-6126, miR-7977, miR-1237-5p, miR-5787, miR-6075, miR-6089, miR-6893-5p, and miR-7704) were lowly expressed in HHSteC cells. The results of RNA expression analysis by microarray of the above-described 11 types of miRNAs in Example 1 and the present Example are shown in Table 2.

TABLE 2

| | Healthy person | | | Liver cirrhosis case | | | HHSteC |
|---|---|---|---|---|---|---|---|
| | EVs | EVs | EVs | EVs | EVs | EVS | Cell |
| hsa- | 3504 | 3562 | 6495 | 5106 | 5712 | 1937 | 0 |
| miR- | | | | | | | |
| 204-3p | | | | | | | |
| hsa- | 2790 | 4196 | 4675 | 3857 | 3104 | 3801 | 21 |
| miR- | | | | | | | |
| 5787 | | | | | | | |
| hsa- | 7041 | 8010 | 13730 | 9911 | 20800 | 19239 | 116 |
| miR- | | | | | | | |
| 3960 | | | | | | | |
| hsa- | 1170 | 1750 | 2388 | 1497 | 3480 | 2328 | 26 |
| miR- | | | | | | | |
| 6075 | | | | | | | |
| hsa- | 1397 | 1832 | 2621 | 3291 | 4564 | 5781 | 29 |
| miR- | | | | | | | |
| 1237-5p | | | | | | | |
| hsa- | 2741 | 3139 | 4919 | 3540 | 7122 | 3788 | 32 |
| miR- | | | | | | | |
| 7977 | | | | | | | |
| hsa- | 1539 | 1777 | 2907 | 2288 | 3965 | 3925 | 37 |
| miR- | | | | | | | |
| 6893-5p | | | | | | | |
| hsa- | 2803 | 3707 | 5629 | 3794 | 8137 | 9941 | 51 |
| miR- | | | | | | | |
| 6089 | | | | | | | |
| hsa- | 2987 | 3564 | 5897 | 3851 | 10352 | 2602 | 59 |
| miR- | | | | | | | |
| 1908-5p | | | | | | | |
| hsa- | 3813 | 4305 | 7585 | 6475 | 12598 | 6222 | 75 |
| miR- | | | | | | | |
| 6126 | | | | | | | |
| hsa- | 3923 | 5637 | 7282 | 6711 | 11345 | 10341 | 82 |
| miR- | | | | | | | |
| 7704 | | | | | | | |

[Example 3] Eleven types of miRNAs that were highly expressed in mesenchymal stem cell (MSC)-derived extracellular vesicles (EVs) and were effective for suppressing fibrosis, were selected by Examples 1 and 2. It was checked whether these miRNAs were expressed in mesenchymal cells induced from iPS cells (hereinafter, also referred to as “iMSC”).

First, human iPS cells were cultured in a partially modified medium based on StemFit For Mesenchymal Stem Cell (Ajinomoto Co., Inc.), and iPS cell-derived mesenchymal stem cells iMSCs were obtained. Next, extracellular vesicles (MSC-EVs) were harvested by a method similar to that of Example 1, and microarray analysis was performed by a method similar to that of Example 2. The results are shown in Table 3.

TABLE 3

| | iMSC |
|---|---|
| | EVs |
| hsa-miR-204-3p | 2291 |
| hsa-miR-5787 | 2580 |
| hsa-miR-3960 | 6581 |
| hsa-miR-6075 | 1133 |
| hsa-miR-1237-5p | 1279 |
| hsa-miR-7977 | 2586 |
| hsa-miR-6893-5p | 2995 |
| hsa-miR-6089 | 3479 |
| hsa-miR-1908-5p | 3766 |
| hsa-miR-6126 | 3361 |
| hsa-miR-7704 | 6089 |

From Table 3, it was verified that the above-described 11 types of microRNAs were highly expressed in the iMSCs as well.

### [Example 4] Selection of miRNAs Effective for Suppressing Fibrosis

It is not clearly known whether the 11 types of miRNAs obtained in Example 2 suppress or enhance fibrosis of HHSteC cells. Thus, it was investigated whether each of the miRNAs has a fibrosis suppressive action. Specifically, a cocktail was prepared by mixing 11 types of miRNAs, and when a group of 10 types of miRNAs prepared by excluding only one type of miRNA from the cocktail showed enhanced fibrosis compared to the group of 11 types of miRNAs, it was judged that the eliminated miRNA has a fibrosis suppressive action, thereby performing selection. Specifically, the selection was performed by the following method.
(1) A miR cocktail (total 10 μmol) was produced by mixing 11 types of miRNAs or 10 types of miRNAs excluding one type from the 11 types in a PBS solution, and 50 μL of DMEM including the miR cocktail was prepared.
(2) 3 μL of Lipofectamin RNAiMax (Thermo Fisher Scientific Inc.) and 50 μL of DMEM were mixed to form a suspension.
(3) Both of the above-described (1) and the above-described (2) were mixed to form a suspension, and the mixture was left at room temperature for 5 minutes or longer.
(4) Hepatic stellate cells (HHSteC) were harvested and activated with transforming growth factor (TGF)-β, and then the cells were seeded at 5×10⁴ cells per well on a 24-well plate (DMEM+10% Exofree FBS; 500 μL+TGF-β (5 μg/mL))
(5) 50 μL each of the suspension that had been left at room temperature in (3) was administered to each of the seeded plates (final concentration; RNA: 5 pmol/well, Lipofectamine: 1.5 μL/well).
(6) The cells were cultured at 37° C. for 2 days, the cultured cells were harvested, and fibrosis markers (ACTA2, COL1A1, and ELN) were evaluated by a real-time-PCR method.

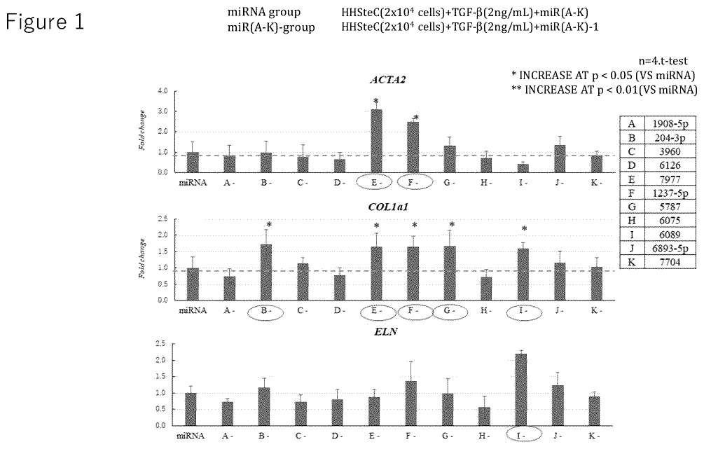

The relative ratio of each of the groups of 10 types of miRNAs obtained when the Fold change in the case of the group of 11 types of miRNAs was taken as 1.0 is shown in FIG. 1. ACTA2 is a gene that is expressed when hepatic stellate cells are activated, COL1A1 is a gene related to fibrosis, while ELN is a major constituent component of elastic fibers, and it can be evaluated that t when suppression of the expression of any one of them is led to suppression of fibrosis.

Based on the results of FIG. 1, B, E, F, G, and I (miR-204-3p, miR-7977, miR-1237-5p, miR-5787, and miR-6089) were selected as candidates for miRNAs effective for suppressing fibrosis.

### [Example 5] Fibrosis Suppressive Action by Selected Five miRNAs

The fibrosis suppressive action in HHSteC was checked using the five miRNAs selected in Example 4.

HHSteC cells were used in the same manner as in Example 4. The cells were divided into the following four groups, and the fibrosis suppressive action of a 5-kind miRNAs group or 4-kind miRNAs groups was investigated.

Control group: HHSteC (2×10⁴ cells)

TGF-β group: HHSteC (2×10⁴ cells)+TGF-β (2 ng/mL)

5-kind miRNAs group: HHSteC (2×10⁴ cells)+TGF-β (2 ng/mL)+miR-1237-5p, miR-204-3p, miR-7977, miR-6089, and miR-5787 (B+E+F+G+I: 0.28 μg each, Total 1.4 μg)

4-kind miRNAs groups: HHSteC (2×10⁴ cells)+TGF-β (2 ng/mL)+4 kinds among miR-1237-5p, miR-204-3p, miR-7977, miR-6089, and miR-5787 (a combination of 4 kinds among B, E, F, G, and I: 0.35 μg each, Total 1.4 μg)

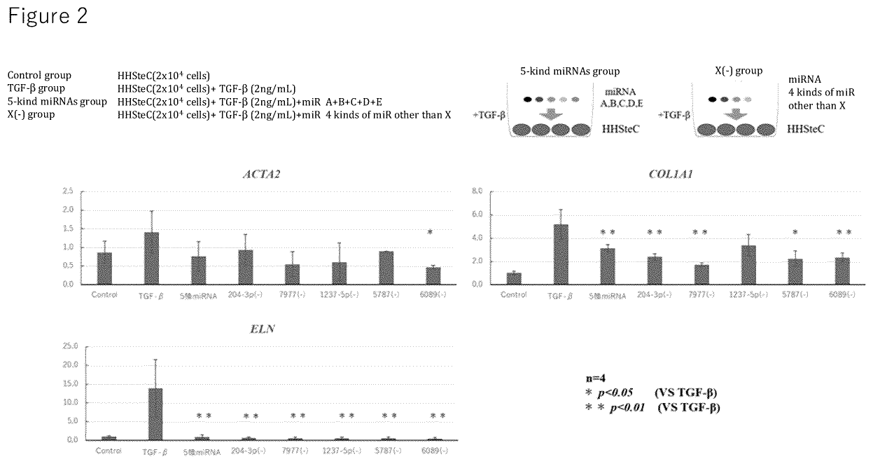

The relative ratios of the TGF-β group, the group of 5 types of miRNAs, and the groups of 4 types of miRNAs when the Fold change of the control group was taken as 1.0 are shown in FIG. 2.

From FIG. 2, the group of 5 types of miRNAs suppressed the expression of any of the genes even in the presence of TGF-β. Particularly, the group of 5 types of miRNAs suppressed the expression of ACTA2 and ELN, which are significantly involved as factors for fibrosis of the liver, even to the level similar to that of the control. In addition, in the case of any of the 4-kind miRNAs, the expression of ACTA2, COL1A1, and ELN was suppressed as compared with the case where only TGF-β was added, and particularly in regard to ELN, the expression was lowered to the same level as that of the control. Therefore, it was verified that fibrosis of the liver can be suppressed by using miR-1237-5p, 204-3p, 7977, 6089, and 5787 singly or in combination.

### [Example 6] Fibrosis Suppressive Action of Each of Selected Five miRNAs Alone

In Example 5, the fibrosis suppressive action of the 5-kind miRNAs group or the 4-kind miRNAs groups among the five miRNAs selected in Example 4 was investigated; however, in this Example, the fibrosis suppressive action in HHSteC was checked using single miRNAs.

HHSteC cells were used in the same manner as in Example 4. The cells were divided into the following eight groups, and the fibrosis suppressive action of the single miRNA groups was investigated.

Control group: HHSteC (2×10⁴ cells)

TGF-β group: HHSteC (2×10⁴ cells)+TGF-β (2 ng/mL)

5-kind miRNAs group: HHSteC (2×10⁴ cells)+TGF-β (2 ng/mL)+miR-1237-5p, miR 204-3p, miR-7977, miR-6089, miR-5787 (0.28 μg each, Total 1.4 μg)

Single miRNA Groups:

$$\begin{matrix}HHSteC ( 2 \times 1 {0}^{4} cells ) + TGF ‐ β ( 2 ng / ML ) + miR 1237 - 5 p ( 1.4 µg ) \\ HHSteC ( 2 \times 1 {0}^{4} cells ) + TGF ‐ β ( 2 ng / ML ) + miR 204 - 5 p ( 1.4 µg ) \\ HHSteC ( 2 \times 1 {0}^{4} cells ) + TGF ‐ β ( 2 ng / ML ) + miR 7977 ( 1.4 µg ) \\ HHSteC ( 2 \times 1 {0}^{4} cells ) + TGF ‐ β ( 2 ng / ML ) + miR 6089 ( 1.4 µg ) \\ HHSteC ( 2 \times 1 {0}^{4} cells ) + TGF ‐ β ( 2 ng / ML ) + miR 5787 ( 1.4 µg )\end{matrix}$$

The fibrosis suppressive action of miRNAs was investigated in the same manner as in Example 4, except that COL1A2 and COL3A1 were evaluated together with ACTA2, COL1A1, and ELN, which are fibrosis markers, by the real-time-PCR method. COL1A2 and COL3A1 are genes related to fibrosis.

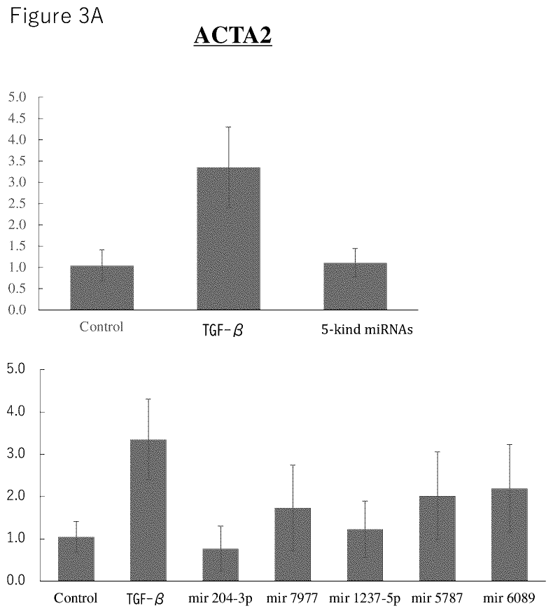

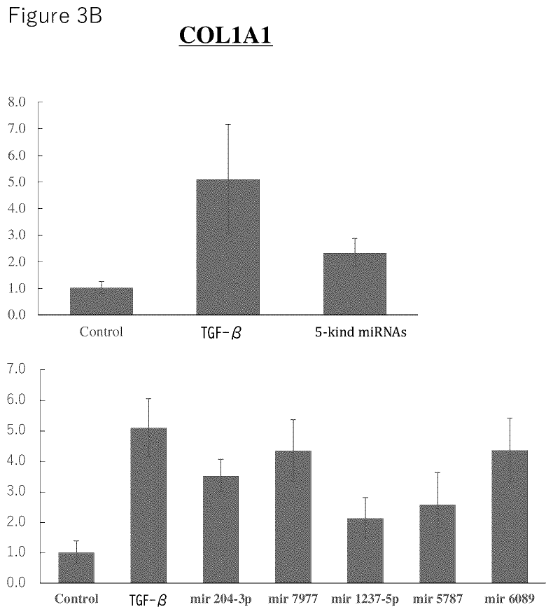

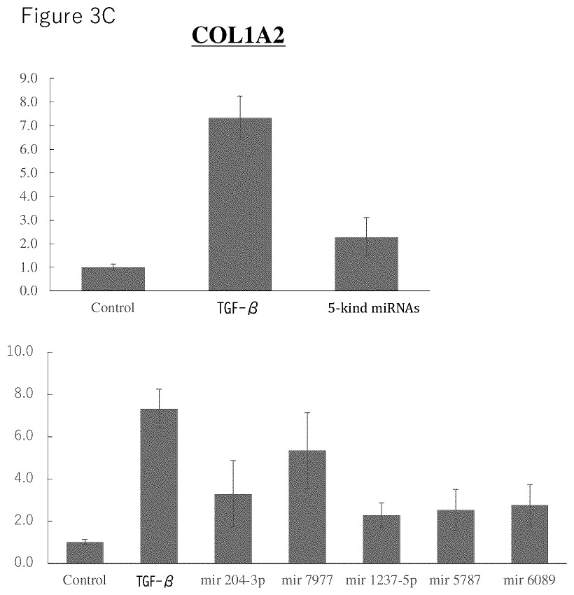

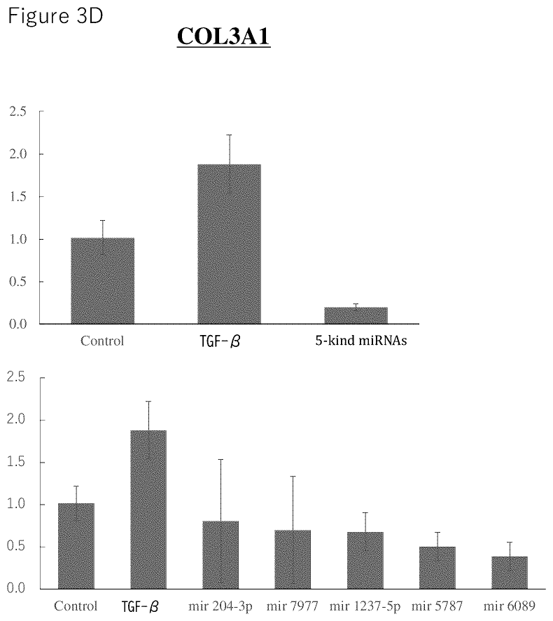

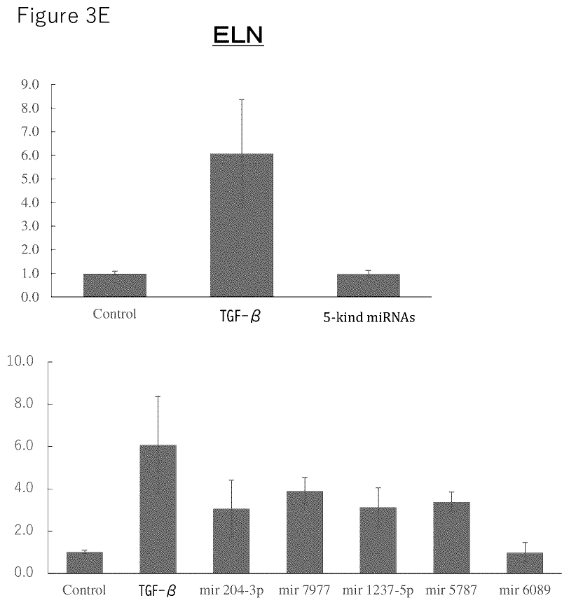

The relative ratio of each of the TGF-β group and the single miRNA groups when the Fold change of the control group was taken as 1.0 is shown in FIG. 3A to FIG. 3E. In FIG. 3A to FIG. 3E, the upper row shows the Control group, the TGF-β group, and the 5-kind miRNAs group, and the lower row shows the Control group, the TGF-β group, and the Single miRNA group.

From FIG. 3A to FIG. 3E, not only the group of 5 types of miRNAs but each of the single miRNA groups of miR 204-3p, miR 7977, miR 1237-5p, miR 5787, and miR 6089 also suppressed the expression of fibrosis marker genes in the presence of TGF-β.

When viewed individually, miR 204-3p decreased the expression of ACTA2 to the level of the control, and also decreased the expression of COL1A2, COL3A1, and ELN to about 50% as compared with the TGF-β group.

miR 7977 decreased the expression of COL3A1 by about 60%, the expression of ACTA2 by about 50%, and the expression of ELN by about 35%, as compared with the TGF-β group.

miR 1237-5p decreased the expression of COL1A2 by 70%, the expression of ACTA2 and COL1A3 by about 60%, and the expression of COL1A1 and ELN by about 50%, as compared with the TGF-β group.

miR 5787 decreased the expression of COL1A2 and COL3A1 by 70%, the expression of COL1A1 by about 50%, and the expression of ACTA2 and ELN by about 40%, as compared with the TGF-β group.

miR 6089 decreased the expression of COL3A1 and ELN by about 80%, and the expression of COL1A2 by about 60%, as compared with the TGF-β group.

From the above-described results, it was verified that fibrosis of the liver can be suppressed not only when miR 1237-5p, miR 204-3p, miR 7977, miR 6089, and miR 5787 are used in combination of the five kinds, but also when they are used singly.

### [Example 7] Fibrosis Suppressive Action of Selected Five miRNAs in Mice

In the above-description, the fibrosis suppressive action was evaluated using HHSteC cells; however, in this Example, fibrosis suppressive action was evaluated using mice.

200 μL each of a solution obtained by mixing corn oil and carbon tetrachloride (CCL4) at a ratio of corn oil:carbon tetrachloride=3:1 was administered into the peritoneum of Coll-GFP mice (male) twice a week for 8 weeks, and liver cirrhosis model mice were produced. In the eighth week, 10 μg of a cocktail of five miRNAs consisting of miR 1237-5p, miR 204-3p, miR 7977, miR 6089, and miR 5787 (15 μg of each miRNA) was intravenously injected twice (5-kind miRNAs group). The control was not administered with the cocktail of five miRNAs (control group).

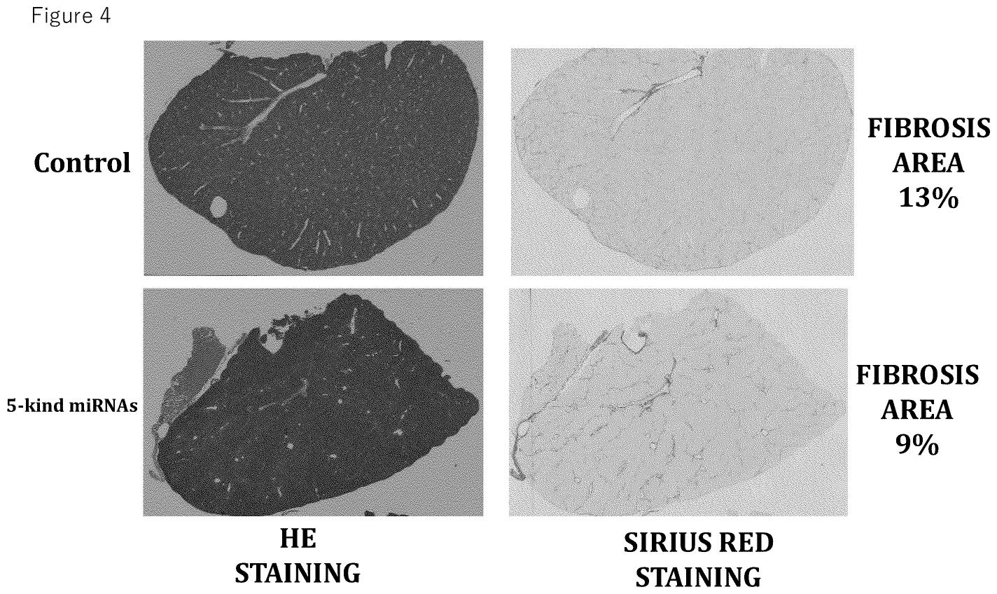

Liver tissues were collected from the mice, the collected liver tissues were immersed in formalin, and paraffin-embedded unstained specimens were produced from the formalin-fixed tissues. The above-described paraffin-embedded unstained specimens were subjected to hematoxylin and eosin (HE) staining and Sirius red staining by standard methods. The results are shown in FIG. 4. The fibrosis area was calculated from the red area of Sirius red. It was verified from the results of HE staining that no fat was attached to any of the tissues.

From FIG. 4, while the fibrosis area of the control group was 13%, the fibrosis area of the group administered with 5 kinds of miRNAs was 9%, and it was verified that the group administered with 5 kinds of miRNAs decreased the fibrosis area more by 30% as compared to the control group. Therefore, it was found that the fibrosis suppressive action of the miRNAs evaluated in the HHSteC cells also showed the same effect in mice.

### [Example 8] Fibrosis Suppressive Action by Administration of Extracellular Vesicles

It was verified by the results of Examples 1 and 2 that five miRNAs consisting of miR 1237-5p, miR 204-3p, miR 7977, miR 6089, and miR 5787 were highly expressed in the mesenchymal stem cell (MSC)-derived extracellular vesicles (EVs). Thus, the fibrosis suppressive action by administering MSC-derived EVs was investigated.

Commercially available extracellular vesicles (EVs) derived from mesenchymal stem cells (MSC) of three healthy persons were harvested in the same manner as in Example 1. Furthermore, HHSteC cells were used in the same manner as in Example 4. The cells were divided into the following three groups, and the fibrosis suppressive action brought by administration of extracellular vesicles was investigated.

Control group: HHSteC (2×10⁵ cells)

TGF-β group: HHSteC (2×10⁴ cells)+TGF-β (2 ng/mL)

Extracellular vesicle-administered group: HHSteC (2×10⁴ cells)+TGF-β (2 ng/mL)+MSC-EVs (2×10⁶ vesicles)

With regard to the fibrosis suppressive action brought by administration of extracellular vesicles, fibrosis markers ACTA2, COL1A1, and ELN were evaluated by the real-time-PCR method.

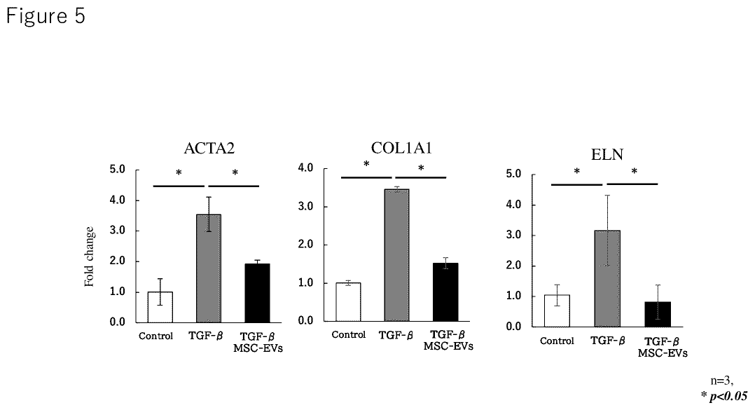

The relative ratio of each of the TGF-β group and the extracellular vesicle-administered group when the Fold change of the control group was taken as 1.0 is shown in FIG. 5.

In FIG. 5, when the MSC-EVs were administered, they decreased the expression of ACTA2 by about 40%, the expression of COL1A1 by about 55%, the expression of ELN by about 80%, and the expression of ELN by about 70%, as compared with the TGF-β group. Therefore, it was verified that not only when miR 1237-5p, miR 204-3p, miR 7977, miR 6089, or miR 5787 is administered, but also when extracellular vesicles containing the above-described microRNAs are administered, fibrosis of the liver can be suppressed.

### SEQUENCE LISTING

R06-036.xml

1. A pharmaceutical composition for preventing or treating a liver disease associated with fibrosis, the pharmaceutical composition comprising at least one kind of microRNAs selected from miR 1237-5p, miR 204-3p, miR 7977, miR 6089, and miR 5787, or precursors thereof as an active ingredient.

2. The pharmaceutical composition according to claim 1, further comprising a vehicle for holding the microRNAs or precursors thereof.

3. The pharmaceutical composition according to claim 2, wherein the vehicle for holding the microRNAs or precursors thereof is any extracellular vesicle selected from an exosome, a microvesicle, a liposome (lipid bilayer), and an apoptotic vesicle.

4. The pharmaceutical composition according to claim 3, wherein the extracellular vesicle is a mesenchymal stem cell-derived extracellular vesicle.

5. The pharmaceutical composition according to claim 3, wherein the extracellular vesicle is a cell-derived extracellular vesicle obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed therein.

6. The pharmaceutical composition according to claim 1, wherein the pharmaceutical composition is used in combination with mesenchymal stem cells to be administered to a target.

7. An extracellular vesicle derived from cells, obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed therein,
the extracellular vesicle comprising at least one kind of microRNAs selected from miR 1237-5p, miR 204-3p, miR 7977, miR 6089, and miR 5787, or precursors thereof.

8. The extracellular vesicle according to claim 7, wherein the extracellular vesicle is a mesenchymal stem cell-derived extracellular vesicle.

9. A culture product obtained by introducing a polynucleotide encoding at least one kind of microRNAs selected from extraneous miR 1237-5p, miR 204-3p, miR 7977, miR 6089, and miR 5787, or precursors thereof into cells, allowing the microRNAs or precursors thereof to be expressed in the cells, and culturing the cells.

10. A method for producing extracellular vesicles for suppressing liver fibrosis, the extracellular vesicles containing at least one kind of microRNAs selected from extraneous miR 1237-5p, miR 204-3p, miR 7977, miR 6089, and miR 5787, or precursors thereof, the method comprising the following steps A to C:
step A of introducing a polynucleotide encoding at least one kind of microRNAs selected from extraneous miR 1237-5p, miR 204-3p, miR 7977, miR 6089, and miR 5787, or precursors thereof into mesenchymal stem cells;
step B of culturing the mesenchymal stem cells obtained in step A to obtain a culture product; and
step C of harvesting extracellular vesicles from the culture product.

11. The pharmaceutical composition according to claim 4, wherein the extracellular vesicle is a cell-derived extracellular vesicle obtained by introducing a polynucleotide encoding at least one kind of microRNAs selected from extraneous miR 1237-5p, miR 204-3p, miR 7977, miR 6089, and miR 5787, or precursors thereof into cells, and allowing the microRNAs or precursors thereof to be expressed therein.

12. The pharmaceutical composition according to claim 2, wherein the pharmaceutical composition is used in combination with mesenchymal stem cells to be administered to a target.
